(19) United States
(12) Patent Application Publication
(10) Pub. No.: US 2001/0041876 A1
(43) Pub. Date: Nov. 15, 2001

(54) Superabsorbent and nonwoven composites for personal care products

(76) Inventors: Christopher Cosgrove Creagan, Marietta, GA (US); Samuel Edward Marmon, Melbourne, FL (US); Debra Jean McDowall, Neenah, WI (US); David Lewis Myers, Cumming, GA (US); Xin Ning, Alpharetta, GA (US)

Correspondence Address: James B. Robinson, Kimberly-Clark Worldwide, Inc., Patent Department, 401 North Lake Street, Neenah, WI, 54956, US

(21) Appl. No.: 09/728,242

(22) Filed: Dec. 1, 2000

Related U.S. Application Data
(60) Provisional application No. 60/173,038, filed on Dec. 23, 1999.

(51) Int. Cl. A61F 13/15

(52) U.S. Cl. 604/368; 604/370

(57) ABSTRACT

There is provided an expandable absorbent material having superabsorbent in an amount from about 1 to about 75 weight percent and crimped thermoplastic fibers in an amount from about 25 to about 99 weight percent, where said material has a density of from about 0.02 g/cc to about 0.25 g/cc. The superabsorbent may be in the form of particulate, fiber and foam. Such a composite may be used in personal care products like diapers, training pants, absorbent underpants, adult incontinence products, and the like.

This application claims priority from U.S. Provisional Application No. 60/173,038 filed Dec. 23, 1999.

### FIELD OF THE INVENTION

This invention relates to absorbent articles, particularly absorbent structures that are useful in personal care products such as disposable diapers, incontinence guards, childcare training and refastenable pants and the like. More particularly, the invention relates to absorbent articles that are thin and conformable and that expand upon insult to provide additional volume into which liquid may be taken.

### BACKGROUND OF THE INVENTION

Personal care absorbent products should have low or no leakage, a dry feel to the wearer, and should be thin so as to be more comfortable to the wearer and less apparent in use. Current absorbent products, however, often fail to meet these objectives for a variety of reasons.

Leakage can occur because of insufficient uptake rate by layers intended to provide retention or distribution capability in the intake or target zone. Attempts to alleviate leakage occurring by this mechanism include absorbent articles that incorporate surge material structures located above (i.e., toward the wearer) the retention or distribution materials. U.S. Pat. No. 5,364,382 to Latimer discloses nonwoven materials such as meltblowns, bonded carded webs, and pulp coforms that receive and subsequently release liquid to the retention means. The material structures of Latimer utilize large denier resilient fibers blended with small denier wettable fibers to achieve rapid liquid uptake and rapid liquid release to the underlying retention storage material. Additionally, U.S. Pat. No. 5,490,846 to Ellis discloses layered structures to improve intake rates of surge materials.

Despite the development of surge materials that attempt to achieve rapid uptake and rapid release to the retention material, the objective of thinness remains to be satisfactorily reached. The cited surge materials are quite thick and when placed into the intake zone of the absorbent article can cause poor fit in the crotch region of the absorbent product upon initial wearing and can lead to several performance problems, or particularly for adult incontinence garment wearers, provide an embarrassing appearance. Firstly, the product can leak due to gapping that is created by the bulky surge material. Secondly, the product is not comfortable to the wearer when a bulky material is utilized to provide the necessary void volume for uptake. There remains a need, therefore, for materials which will rapidly uptake an insult to the target area and either store the insult or release it for subsequent storage, and which also remain relatively thin prior to insult.

It is an object of this invention to provide a material for personal care products which rapidly uptakes an insult and which can store it or transfer it to an adjacent material for distribution or storage, and which remains relatively thin prior to such insult. It is another object of this invention to provide a personal care product that, prior to insult, is thin and comfortable for a wearer.

### SUMMARY OF THE INVENTION

The objectives of the invention are achieved by materials and products which have been designed to be very thin prior to insult and expand rapidly when insulted. The invention is a relatively low density, expandable absorbent composite material made from superabsorbent material in an amount from about 1 to about 75 weight percent and crimped nonwoven fibers in an amount from about 25 to about 99 weight percent, where the material has a density of from about 0.02 g/cc to about 0.25 g/cc. The nonwoven fibers are preferably conjugate or bicomponent fiber.

Such a composite may be used in personal care products like diapers, refastenable and training pants, absorbent underpants, feminine hygiene products, adult incontinence products, and the like.

### BRIEF DESCRIPTION OF THE DRAWINGS

FIG. 1 is a cradle used in the MIST evaluation test as described herein.

### DEFINITIONS

“Layer” when used in the singular can have the dual meaning of a single element or a plurality of elements.

“Liquid” means a nongaseous, nonparticulate substance and/or material that flows and can assume the interior shape of a container into which it is poured or placed.

As used herein the term “nonwoven fabric or web” means a web having a structure of individual fibers or threads, which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

“Spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and usually, though not always, have average diameters (from a sample of at least 10) larger than 7 microns.

“Conjugate fibers” refers to fibers which have been formed from at least two polymer sources extruded from separate extruders but spun together to form one fiber. Conjugate fibers are also sometimes referred to as multicomponent or bicomponent fibers. The polymers are usually different from each other though conjugate fibers may be monocomponent fibers. The polymers are arranged in substantially constantly positioned distinct zones across the cross-section of the conjugate fibers and extend continuously along the length of the conjugate fibers. The configuration of such a conjugate fiber may be, for example, a sheath/core arrangement wherein one polymer is surrounded by another or may be a side by side arrangement, a pie arrangement or an “islands-in-the-sea” arrangement. For two component fibers, the polymers may be present in ratios of 75/25, 50/50, 25/75 or any other desired ratios. The fibers may also have shapes such as those described in U.S. Pat. No. 5,277,976 to Hogle et al., which describes fibers with unconventional shapes.

The methods for making conjugate fibers are well known and need not be described herein in detail. To form a conjugate fiber, generally, two polymers are extruded separately and fed to a polymer distribution system where the polymers are introduced into a segmented spinneret plate. The polymers follow separate paths to the fiber spinneret and are combined in a spinneret hole which to provide the desired polymer arrangement. One example comprises either two or more concentric circular holes thus providing a sheath/core type fiber or a circular spinneret hole divided along a diameter into two parts to provide a side-by-side type fiber. The combined polymer filament is then cooled, solidified and drawn, generally by a mechanical rolls system, to an intermediate filament diameter and collected. Subsequently, the filament may be “cold drawn”, at a temperature below its softening temperature, to the desired finished fiber diameter. Conjugate fibers can be cut into relatively short lengths, such as staple fibers, which generally have lengths in the range of 25 to 51 millimeters (mm), and short-cut fibers, which are even shorter and generally have lengths less than 18 millimeters. See, for example, U.S. Pat. No. 4,789,592 to Taniguchi et al. and U.S. Pat. No. 5,336,552 to Strack et al, both of which are incorporated herein by reference in their entirety.

Conjugate fibers are taught, for example, in U.S. Pat. No. 5,382,400 to Pike et al. and may be crimped by using the differential rates of expansion and contraction of the two (or more) polymers. Such fibers may also be splittable. Crimped fibers may be produced from conjugate or monocomponent fibers by mechanical means as well. A still further method of producing crimped fibers is through the use of “crimp enhancing additives”. Such additives may be added to one of the polymeric components of the conjugate fiber in an amount of from about 0.5 weight percent to about 10 weight percent, more preferably about 5 weight percent. A butylene-propylene copolymer has been found to function particularly well as a crimp enhancing additive. Such a copolymer is preferably random and should have less than about 20 weight percent butylene, more particularly about 14 weight percent butylene. A commercially available polymer that functions as a crimp enhancing additive is marketed by the Union Carbide Corporation as DS4D05 and is a butylene-propylene random copolymer having 14 weight percent butylene and 86 weight percent polypropylene. Further discussion of crimp enhancing additives may be found in U.S. patent application Ser. No. 08/940,886.

As used herein, through-air bonding or “TAB” means a process of bonding a nonwoven conjugate fiber web in which air which is sufficiently hot to melt one of the polymers of which the fibers of the web are made is forced through the web. The air velocity is usually between 100 and 500 feet per minute and the dwell time may be as long as 6 seconds, for example. The melting and resolidification of the polymer provides the bonding. In the through-air bonder, air having a temperature above the melting temperature of one component and below the melting temperature of another component is directed from a surrounding hood, through the web, and into a perforated roller supporting the web. Alternatively, the through-air bonder may be a flat arrangement wherein the air is directed vertically downward onto the web. The operating conditions of the two configurations are similar, the primary difference being the geometry of the web during bonding. The hot air melts the lower melting polymer component and thereby forms bonds between the filaments to integrate the web.

“Bonded carded web” refers to webs that are made from staple fibers which are sent through a combing or carding unit, which breaks apart and aligns the staple fibers in the machine direction to form a generally machine direction-oriented fibrous nonwoven web. Such fibers are usually purchased in bales, which are placed in a picker, which separates the fibers prior to the carding unit. Once the web is formed, it then is bonded by one or more of several known bonding methods. One such bonding method is powder bonding, wherein a powdered adhesive is distributed through the web and then activated, usually by heating the web and adhesive with hot air. Another suitable bonding method is pattern bonding, wherein heated calender rolls or ultrasonic bonding equipment are used to bond the fibers together, usually in a localized bond pattern, though the web can be bonded across its entire surface if so desired. Another suitable and well-known bonding method, particularly when using conjugate staple fibers, is through-air bonding.

“Airlaying” is a well-known process by which a fibrous nonwoven layer can be formed. In the airlaying process, bundles of small fibers having typical lengths ranging from about 2 to about 19 millimeters (mm) are separated and entrained in an air supply and then deposited onto a forming screen, usually with the assistance of a vacuum supply. The randomly deposited fibers then are bonded to one another using, for example, hot air or a spray adhesive.

“Personal care product” means diapers, refastenable and training pants, absorbent underpants, feminine hygiene products and adult incontinence products.

### Test Methods

Material Caliper (Thickness):

The caliper of materials, which is a measure of thickness, is measured at 0.05 psi with a Starret-type bulk tester, in units of millimeters (mm). Three measurements are generally averaged to arrive at a final caliper.

Density:

The density of the materials is calculated by dividing the weight per unit area of a sample in grams per square meter (gsm) by the caliper of the sample in millimeters at 0.05 pi (68.9 Pascals) and multiplying the result by 0.001 to convert the value to grams per cubic centimeter (g/cc). A total of three samples would be evaluated and averaged for the density values.

Multiple Insult Test (MIST Evaluation):

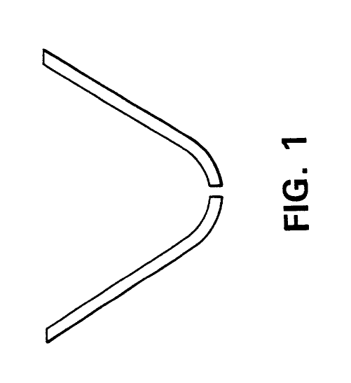

In this test a material is placed in an acrylic cradle to simulate body curvature of a user such as an infant. Such a cradle is illustrated in FIG. 1. The cradle has a width into the page of the drawing as shown of 33 cm and the ends are blocked off, a height of 19 cm, an inner distance between the upper arms of 30.5 cm and an angle between the upper arms of 60 degrees. The cradle has a 6.5 mm wide slot at the lowest point running the length of the cradle into the page.

The material to be tested is placed on a piece of polyethylene film the same size as the sample and placed in the cradle. The material to be tested is insulted with a predetermined amount of a bloodbank saline solution of 1 weight percent of sodium chloride, at a predetermined rate with a nozzle normal to the center of the material and ¼ to ½ inch (6.4 to 12.7 mm) above the material. The amount of runoff is recorded. The material is immediately removed from the cradle, weighed, and placed on a dry 40/60 pulp/superabsorbent pad having a density of 0.2 g/cc in a horizontal position under 0.05 psi pressure and weighed after 15 minutes to determine liquid desorption from the material into the superabsorbent pad as well as liquid retention in the material. The pulp fluff and superabsorbent used in this test is Coosa River's (of Alabama) CR-2054 pulp and Stockhausen Company's (of Greensboro, N.C. 27406) FAVOR® 870 superabsorbent though other comparable pulp and superabsorbents could be used provided they yield a desorption pad of 500 gsm and 0.2 g/cc which after immersion into saline solution under free-swell conditions for 5 minutes, retains at least 20 grams of saline solution per gram of desorption pad after being subjected to a pressure of about 0.5 psi (about 3.45 kPa) for 5 minutes. This test is repeated using fresh desorption pads on each insult so that a total of three insults are introduced.

### Detailed Description of the Invention

The structures of this invention have been designed to be thin materials that expand rapidly when insulted. Therefore, when in use, an absorbent article containing the invention is very thin and comfortable and facilitates good fit. As the material of the invention expands during use, it creates void volume to accommodate incoming fluid, which in turn reduces leakage.

The materials of this invention may desirably function as intake or surge materials in a personal care product. They may alternatively serve as the retention component of a personal care product, depending on the amount of superabsorbent added to the crimped fibers and the amount of expansion desired.

Surge control materials are provided to quickly accept the incoming insult and either absorb, hold, channel or otherwise manage the liquid so that it does not leak outside the article. A surge material must typically be capable of handling an incoming insult of between about 60 and 100 cc at an insult volumetric flow rate of from about 5 to 20 cc/sec, for infants, for example.

Retention materials must absorb the insult efficiently and retain it under pressure. They should be capable of pulling the liquid from the layers nearer the body of the wearer and absorbing the liquid without significant “gel blocking” or blocking of penetration of liquid further into the absorbent by the expansion of the outer layers of absorbent. Retention materials often contain high rate superabsorbent materials such as blends of polyacrylate superabsorbent and fluff. These materials rapidly absorb and hold liquid.

The material of the invention is made from thermoplastic fibers and superabsorbent. The fibers must be crimped and this may be accomplished before or after the formation of the fibers into a coherent nonwoven web. The purpose of using crimped or crimpable fibers in the practice of this invention is to provide a structure into or onto which superabsorbent may be added and which will allow for expansion of the superabsorbent upon wetting. Crimped fibers provide the necessary flexibility and fiber spacing to a web to allow the superabsorbent to expand and yet maintain web integrity and some amount of void volume.

Fibers may be crimped to varying degrees. The crimping of fibers is generally measured as crimps per inch (cpi) or crimps per centimeter (cpcm). The degree of crimping desired in the fibers used to practice this invention is between about 3 cpi (1.2 cpcm) and 100 cpi (39 cpcm). More particularly, a crimp rate of 5 to 25 cpi (2 to 10 cpcm) is desirable and still more particularly a crimp rate of 5 to 15 cpi (2 to 6 cpcm) is beneficial.

The amount of superabsorbent material used in this invention is from about 1 to about 75 weight percent, more particularly between 20 and 60 weight percent and still more particularly between 30 and 50 weight percent. The crimped nonwoven fibers may be present in an amount from about 25 to about 99 weight percent, more particularly between 40 and 80 weight percent and still more particularly between 50 and 70 weight percent. The material has a density of from about 0.02 g/cc to about 0.25 g/cc, or more particularly between 0.02 and 0.15 g/cc. The nonwoven fibers are preferably conjugate or bicomponent fiber. When the material of this invention is used as a retention material, it may desirably have superabsorbent in an amount between about 50 and 75 weight percent, crimped fibers in an amount of about 25 to 50 weight percent, and a density between about 0.05 and 0.25 g/cc.

Thermoplastic fibers are made from, for example, polyolefins, polyamides, rayon, acrylics, poly (vinyl alcohol), polyethylene terephthalate, LYOCELL® regenerated cellulose and elastomeric thermoplastic polymers like those made from block copolymers such as polyurethanes, copolyether esters, polyamide polyether block copolymers, ethylene vinyl acetates (EVA), and block copolymers having styrenic moieties.

Thermoplastic fibers may also be produced from polymers made using metallocene or other single site catalysts. Such polymers are available from Exxon Chemical Company of Baytown, Tex. under the trade name ACHIEVE® for polypropylene-based polymers and EXACT® and EXCEED® for polyethylene based polymers. Dow Chemical Company of Midland, Mich. has polymers commercially available under the name ENGAGE®.

Many polyolefins are available for fiber production, for example polyethylenes such as Dow Chemical's ASPUN® 6811A linear low-density polyethylene, 2553 LLDPE and 25355 and 12350 high-density polyethylene are such suitable polymers. The polyethylenes have melt flow rates, respectively, of about 26, 40, 25 and 12. Fiber forming polypropylenes include Exxon Chemical Company's Escorene® PD 3445 polypropylene and Himont Chemical Co.'s PF-304. Many other polyolefins are commercially available. Other classes of polymer may be used; such as a water-degradable poly (vinyl alcohol) is available as AX2000 from Nippon Synthetic Chemical Company, Ltd. of Osaka, Japan.

Superabsorbents that are useful in the present inventions can be chosen from classes based on chemical structure as well as physical form. These include superabsorbents with low gel strength, high gel strength, surface cross-linked superabsorbents, uniformly cross-linked superabsorbents, or superabsorbents with varied cross-link density throughout the structure. Superabsorbents may be based on chemistries that include but are not limited to acrylic acid, iso-butylene/maleic anhydride, polyethylene oxide, carboxy-methyl cellulose, poly (vinyl pyrrollidone), and poly (vinyl alcohol). The superabsorbents may range in rate from slow to fast. The superabsorbents may be in the form of foams, macroporous or microporous particles or fibers, may have fuzzy or fibrous coatings or morphology. The superabsorbents may also be in the shape of ribbons, particles and may also be in fibrous form. If the superabsorbent is in fibrous form it may also be a part of the conjugate fiber used in the practice of the invention such that no additional superabsorbent need be added, beyond that present in the conjugate fiber. Superabsorbents may be in various length and diameter sizes and distributions. The superabsorbents may be in various degrees of neutralization. Neutralization occurs through use of counter ions such as Li, Na, K, Ca.

Materials of this invention may include superabsorbents of the types mentioned above. One producer of superabsorbents is The Dow Chemical Company of Midland, Mich. Another producer of superabsorbents is Stockhausen, Inc. which produces FAVOR® 880 superabsorbent. Superabsorbents obtained from Camelot are designated Fiberdri 1241 and Fiberdri 1161. Superabsorbents obtained from Technical Absorbents, Ltd. are designated Oasis 101 and Oasis 111. Another example included in these types of superabsorbents is obtained from Chemdall and is recognized as Flosorb 60 Lady. Another example included in these types of superabsorbents is obtained from Sumitomo Seika and is recognized as SA60N Type 2. Additional types of superabsorbents not listed here which are commonly available and known to those skilled in the art can also be useful in the present invention.

Binders typically used in these structures help provide mechanical integrity and stabilization and may also be used to adhere superabsorbent to the thermoplastic fibers. Binders include fiber, liquid or other binder means, which may be thermally activated. Preferred fibers for inclusion are those that will crimp and also bind the web together such as conjugate fibers. Lower melting point polymers in the conjugate fiber provide the ability to bond the fabric together at fiber crossover points upon the application of heat as in through air bonding. Fibers having a lower melting polymer are generally referred to as “fusible fibers”. By “lower melting polymers” what is meant are those having a melting temperature less than about 175 C. It should be noted that the texture of the absorbent web could be modified from soft to stiff through selection of the glass transition temperature of the polymer. Exemplary binder fibers include conjugate fibers of polyolefins, polyamides and polyesters. Three suitable binder fibers are sheath core conjugate fibers available from KoSa Inc. (Charlotte, N.C.) under the designation T-255 and T-256 or Copolyester designation, though many suitable binder fibers are known to those skilled in the art, and are available by many manufacturers such as Chisso and Fibervisions LLC of Wilmington, Del. KoSa has developed a suitable co-polyester binder fiber as a sheath core application and is known by designation T254 (low melt copolyester).

Liquid binders may also be used, particularly in cases in which the fibers are mechanically crimped or when the conjugate fibers crimp but do not have a suitable fusible polymer included. A suitable liquid binder is KYMENE® 557LX available from Hercules Inc. of Wilmington, Del. Other suitable liquid binders include ethylene vinyl acetate emulsion polymers sold by National Starch and Chemical Company (Bridgewater, N.J.) under the tradename Dur-O-Set®) ELITE® series (including ELITE® 33 and ELITE® 22). Air Products Polymers and Chemicals sell other suitable binders under the name AIRFLEX®.

Moisture sensitive binders are available in powder, liquid, or fibrous form that may be activated using heat and/or small amounts of moisture. Binding systems may be poly (vinyl alcohol) adhesives, powders or fibers that dissolve in fluids. Some specific examples of poly (vinyl alcohol)s have easily reversible crosslinks that allow changes in the adhesive property upon contact with the insult allowing the resilient structure to expand. Water sensitive hot melt adhesives could also be used that have time triggers based on controlled hydrophilicity or water triggerable polymers could be used such as base sensitive acrylics. Binders also include polyacrylic amides, polyacrylic acid and its copolymers, starch binders, cellulosic binders, and protein based binders.

The material of this invention may be treated with a selected amount of surfactant, as known to those skilled in the art, such as AHCOVEL®, GLUCOPON®, PLURONICS®, TRITON®, and MASIL SF-19® surfactant, or mixtures thereof, or otherwise processed to impart the desired level of wettability and hydrophilicity. If a surfactant is used, it can be an internal additive or applied to a layer by any conventional means, such as spraying, brush coating and the like.

Electret treatment further increases the capture of superabsorbent onto a web by drawing particles toward the web by virtue of their electrical charge. Such treatment may be used in place of or in conjunction with the use of binders to adhere the superabsorbent to the web.

Electret treatment can be carried out by a number of different techniques. One technique is described in U.S. Pat. No. 5,401,446 to Tsai et al. assigned to the University of Tennessee Research Corporation and incorporated herein by reference in its entirety. Tsai describes a process whereby a web or film is sequentially subjected to a series of electric fields such that adjacent electric fields have substantially opposite polarities with respect to each other. Thus, one side of the web or film is initially subjected to a positive charge while the other side of the web or film is initially subjected to a negative charge. Then, the first side of the web or film is subjected to a negative charge and the other side of the web or film is subjected to a positive charge. Such webs are produced with a relatively high charge density without an attendant surface static electrical charge. The process may be carried out by passing the web through a plurality of dispersed non-arcing electric fields, which may be varied over a range depending on the charge desired in the web. The web may be exposed to a field having a range of about 1 kVDC/cm to 12 kVDC/cm or more particularly 4 kVDC/cm to 10 kVDC/cm and still more particularly 7 kVDC/cm to about 8 kVDC/cm.

Other methods of electret treatment are known in the art such as that described in U.S. Pat. No. 4,215,682 to Kubik et al, U.S. Pat. No. 4,375,718 to Wadsworth, U.S. Pat. No. 4,592,815 to Nakao, U.S. Pat. No. 4,874,659 to Ando and U.S. Pat. No. 5,834,384 to Cohen et al.

Another method of imparting a charge to a nonwoven web is to incorporate charging devices into air baffles or air dams located above the fiber draw unit (FDU) in a spunbond process. The FDU is located just downstream of the fiber quenching area, which is located immediately after fiber formation. As known to those skilled in the art, air dams are typically used in the spunbond manufacturing process to help control the flow of air into the FDU. Air dams fabricated from electrical grade polytetrafluoroethylene may be fitted with charge bar(s) or aluminum bias rod(s). The charge bars may be powered with any suitable high voltage power supply set to deliver a positive potential. The bias bar is grounded to the earth. The air dams are arranged such that the charge bars are on the upstream side of the FDU and the bias bar on the downstream side though this may be reversed if desired. An air gap of about 2.54 cm (1 inch) should be maintained between the two air dams. During fiber spinning the charge bars should be maintained at a point just below the onset of arcing between the bars, usually about 10 kV to 24 kV.

In an experiment to determine the amount of attractiveness of the fibers due to electret charging, FAVOR® 880 superabsorbent particles were sprayed onto the fibers just after discharge from the FDU fitted with the charging system as described above, and before nonwoven web formation. The charged fiber web thus produced contained between 39 and 43 weight percent of FAVOR® 880 particles, expressed as a percentage of web weight. As a control, the same procedure was tested without electrical charging and the web was found to contain only 22 weight percent of the FAVOR®) 880 particles. Electrostatic or electret charging clearly increases the retention of particulates on a nonwoven web.

The following represent various prophetic examples the material of the invention. A crimp amount of about 10 cpi (4 cpcm) is assumed in all fibers that are crimped.

### EXAMPLE 1

Polypropylene/polyethylene side-by-side conjugate fibers may be produced according to the spunbond process and crimped as they are produced by the application of differential cooling as taught in Pike (U.S. Pat. No. 5,382,400) or through the use of a crimp enhancing additive as taught above. Superabsorbent particles and the resulting spunbond web could be placed into a tumbler. The superabsorbent particles could be integrated into the web by tumbling and shaking the excess particles off the web. The web, especially when still warm from the production process, will hold the particles well.

### EXAMPLE 2

Polypropylene monocomponent fibers may be produced according to the spunbond process and mechanically crimped after production. A liquid binder could be sprayed onto the web and superabsorbent particles could be added to the web by tumbling it onto the web and shaking off the excess.

### EXAMPLE 3

Polypropylene/superabsorbent side-by-side conjugate fibers may be produced according to the spunbond process and crimped as they are produced by the application of differential cooling as taught in Pike (U.S. Pat. No. 5,382,400) or through the use of a crimp enhancing additive as taught above. No additional processing steps would be necessary.

### EXAMPLE 4

Polypropylene/polyethylene side-by-side conjugate fibers may be produced according to the spunbond process and crimped as they are produced by the application of differential cooling as taught in Pike (U.S. Pat. No. 5,382,400) or through the use of a crimp enhancing additive as taught above. The web may be electret treated by any of the methods described above and superabsorbent particles sprayed onto the web in an on-line process. A binder may optionally be used.

### EXAMPLE 5

Polypropylene/poly (vinyl alcohol) side-by-side conjugate fibers may be produced according to the spunbond process and crimped as they are produced by the application of differential cooling as taught in Pike (U.S. Pat. No. 5,382,400) or through the use of a crimp enhancing additive as taught above. Superabsorbent particles could be added to the web by tumbling it onto the web and shaking off the excess. The superabsorbent particles may be adhered to the web with a binder, electret treatment or both. Such a material, because of the water solubility of the poly (vinyl alcohol) polymer, may be disposed of by flushing in a toilet.

### EXAMPLE 6

Polypropylene/polyethylene side-by-side conjugate fibers may be produced according to the spunbond process and crimped after they are produced by the application of heat in a through air bonder. The web may be electret treated by any of the methods described above and superabsorbent particles could be added to the web by tumbling it onto the web and shaking off the excess. A binder may optionally be used.

### EXAMPLE 7

Polypropylene fibers may be produced by meltspinning and cut into staple fibers. The polypropylene fibers and superabsorbent fibers may be blended together in the airlaying or bonding and carding processes, stabilized with a binder if necessary, and the resultant web mechanically crimped.

### EXAMPLE 8

Polypropylene fibers may be produced by meltspinning and cut into staple fibers. The polypropylene fibers and superabsorbent fibers may be layered one on top of the other using the airlaying or bonding and carding processes, stabilized with a binder if necessary, and the resultant web mechanically crimped.

In addition to the above prophetic examples, actual examples were produced. In each example, and a control, the thermoplastic fibers had about 5 weight percent of Union Carbide's DS4D05 and were 1 denier polypropylene/polyethylene side-by-side conjugate spunbond fibers having more than 3 cpi. Superabsorbent particles (SAP) were added by tumbling it onto the spunbond web and shaking off the excess. The materials were compressed and then insulted with 60 ml of 10 percent saline solution at 30 minute intervals and the change in thickness reported as (wet thickness−dry thickness)/dry thickness×100, after the third insult. The results are below.

| | SAP Wt. | Basis Wt. | Dry Thick- | Density | Thickness |
|---|---|---|---|---|---|
| | Percent | (gsm) | ness (mm) | (g/cc) | Change (%) |
| Control | 0 | 233 | 6.5 | 0.036 | −19 |
| Example | 16 | 209 | 5.0 | 0.042 | 6 |
| A | | | | | |
| Example | 33 | 255 | 4.2 | 0.06 | 65 |
| B | | | | | |
| Example | 33 | 235 | 2.7 | 0.087 | 101 |
| C | | | | | |

It is clear from the percent thickness change data from the above table that the materials of this invention did successfully expand and so increase the available void volume. While it is not a requirement of the invention, it is preferred that the materials of this invention expand at least about 50 percent in thickness from their compressed dry thickness. In addition, it was noted that the materials were softer and more flexible, providing additional consumer benefits.

In additional testing, materials were subjected to the MIST evaluation test described above using three liquid insults of 80 ml each delivered in 4.5 seconds. The amount of superabsorbent (SAM) in each layer was varied and the overflow or runoff amount, amount of fluid held and thickness were recorded.

The table below contains the data where #1 refers to an 82 gsm, 3.1 dpf crimped polypropylene/polyethylene spunbond fiber treated with 3 weight percent of a 3 to 1 blend of Ahcovel/Glucopon wetability additive, and #2 refers to an 87 gsm mixed fiber size (0.9 and 2.8 dpf) crimped polypropylene/polyethylene spunbond fiber treated with the same amount of the same additive. In the table, the “average” readings are an average of three insults.

| | 2 | #1 over | 2 layers | 2 | #2 over | SAM and |
|---|---|---|---|---|---|---|
| | Layers | #1 + | #1 + | Layers | #2 + | pulp |
| Material | #1 | SAM | SAM | #2 | SAM | fluff |
| Sample | 1.88 | 2.42 | 2.89 | 1.97 | 2.46 | 2.80 |
| weight - g | | | | | | |
| Sample basis | 167 | 214 | 256 | 174 | 218 | 289 |
| weight - gsm | | | | | | |
| Top layer | 0 | 0 | 31 | 0 | 0 | N/A |
| % SAM | | | | | | |
| Bottom layer | 0 | 34 | 36 | 0 | 28 | 40 |
| % SAM | | | | | | |
| 1st Insult | 46.3 | 44.0 | 44.3 | 44.1 | 42.9 | 58.2 |
| Overflow - g | | | | | | |
| 2nd Insult | 46.3 | 43.8 | 43.4 | 44.9 | 41.0 | 56.0 |
| Overflow - g | | | | | | |
| 3rd Insult | 45.6 | 43.5 | 43.1 | 45.1 | 39.4 | 59.3 |
| Overflow - g | | | | | | |
| Avg. | 46.0 | 43.8 | 43.6 | 44.7 | 41.1 | 57.8 |
| Overflow - g | | | | | | |
| Avg. Fluid | 34.3 | 36.0 | 36.6 | 34.9 | 37.7 | 23.3 |
| Held - g | | | | | | |
| Avg. Fluid | 18.4 | 14.9 | 12.7 | 17.7 | 15.3 | 8.2 |
| Held - g/g | | | | | | |
| Avg. Fluid | 0.45 | 1.89 | 3.44 | 0.16 | 2.03 | |
| Retained - | | | | | | |
| g/g | | | | | | |
| Dry Thick- | 273 | 300 | 310 | 248 | 250 | 248 |
| ness - mil | | | | | | |
| Wet Thick- | 251 | 295 | 335 | 227 | 270 | 207 |
| ness - mil | | | | | | |

The data show that the crimped fiber layers having superabsorbent functioned on a par with those that did not contain superabsorbent in regard to intake or surge functions (overflow), yet they retained a much greater amount of the fluid which entered the materials (fluid retained). This may be compared with an airformed composite of superabsorbent and pulp fluff which, as shown in the final column on the right. The data in the right hand column is for an average of four samples of a single layer composite of a blend of 40 weight percent Stockhausen FAVOR® 880 superabsorbent and 60 weight percent Weyerhaeuser CF-416 pulp fluff tested in the same manner as the other samples. A composite of superabsorbent and fluff also has quite poor structural integrity and low flexibility, whereas the instant invention has very good flexibility and integrity. The flexibility of the invention versus that of superabsorbent/fluff composites may be illustrated in an edge-wise compression test in which the materials of the invention perform much better than the superabsorbent/fluff composites. The material of this invention thus combines the attractive features of each type composite without the undesirable drawbacks.

Although only a few exemplary embodiments of this invention have been described in detail above, those skilled in the art will readily appreciate that many modifications are possible in the exemplary embodiments without materially departing from the novel teachings and advantages of this invention.

What is claimed is:

1. An expandable absorbent material comprising superabsorbent in an amount from about 1 to about 75 weight percent and crimped thermoplastic fibers in an amount from about 25 to about 99 weight percent, where said material has a density of from about 0.02 g/cc to about 0.25 g/cc.

2. The material of claim 1 wherein said thermoplastic fibers are conjugate fibers.

3. The material of claim 1 wherein said thermoplastic fibers are elastic fibers.

4. The material of claim 2 wherein said conjugate fibers are comprised of polyolefin.

5. The material of claim 2 wherein said conjugate fibers are comprised of polyolefin and superabsorbent in a side-by-side configuration.

6. The material of claim 2 wherein said conjugate fibers are comprised of polyolefin and poly (vinyl alcohol) in a side-by-side configuration.

7. The material of claim 1 further comprising a binder

8. The material of claim 7 wherein said binder is a liquid.

9. The material of claim 1 wherein said thermoplastic fibers are mechanically crimped.

10. The material of claim 1 wherein said material has an electret charge.

11. The material of claim 1 which expands at least about 50 percent upon wetting.

12. A personal care product selected from the group consisting of diapers, training pants, feminine hygiene products, absorbent underpants and adult incontinence products comprising the material of claim 1.

13. The product of claim 12 wherein said personal care product is an adult incontinence product.

14. The product of claim 12 wherein said personal care product is a feminine hygiene product.

15. The product of claim 12 wherein said personal care product is a diaper.

16. A surge material for personal care products comprising an expandable absorbent material comprising superabsorbent particles in an amount from about 20 to about 60 weight percent and crimped conjugate thermoplastic fibers in an amount from about 40 to about 80 weight percent, where said material has a density of from about 0.02 g/cc to about 0.15 g/cc.

17. The surge material of claim 16 having superabsorbent particles in an amount from about 30 to about 50 weight percent.

18. The material of claim 17 wherein said conjugate fibers are comprised of polyolefin.

19. A retention material for personal care products comprising an expandable absorbent material comprising superabsorbent particles in an amount from about 25 to about 75 weight percent and crimped conjugate thermoplastic fibers in an amount from about 25 to about 50 weight percent, where said material has a density of from about 0.05 g/cc to about 0.25 g/cc.

20. The material of claim 19 wherein said conjugate fibers are comprised of polyolefin.
